(19) United States
(12) Patent Application Publication
(10) Pub. No.: US 2008/0170658 A1
(43) Pub. Date: Jul. 17, 2008

(54) SYSTEM AND METHOD FOR CT IMAGING WITH INCREASED SAMPLING AND REDUCED ARTIFACTS

(76) Inventors: Jed Douglas Pack, Schenectady, NY (US); Michael Harsh, Clifton Park, NY (US); Bruno K.B. De Man, Clifton Park, NY (US)

Correspondence Address: GENERAL ELECTRIC COMPANY;GLOBAL RESEARCH, PATENT DOCKET RM. BLDG. K1-4A59, NISKAYUNA, NY, 12309, US

(21) Appl. No.: 11/622,240

(22) Filed: Jan. 11, 2007

(51) Int. Cl. A61B 6/00

(52) U.S. Cl. 378/20

(57) ABSTRACT

A system and method for increasing apparent axial sampling resolution include acquiring CT data from scans which are offset due to a scan subject motion. Thus, in certain embodiments, a first scan or gantry rotation may be performed, followed by a subject shift. A second scan or gantry rotation may then be performed to acquire overlapping data which is offset from the data of the first scan. The system and method may also be incorporated in helical scanning techniques. By increasing the number of axial samples, some embodiments provide for reduced aliasing artifacts and improved x-ray interlacing.

### BACKGROUND OF THE INVENTION

The present invention relates generally to diagnostic imaging and, more particularly, to a method and apparatus for increasing sampling resolution. By controlling a subject motion in an axial direction during a scan sequence, an increased apparent number of axial samples may be acquired.

Typically, in computed tomography (CT) imaging systems, an x-ray source emits a fan-shaped beam toward a subject or object, such as a patient or a piece of luggage. Hereinafter, the terms “subject” and “object” shall include anything capable of being imaged. The beam, after being attenuated by the subject, impinges upon an array of radiation detectors. The intensity of the attenuated beam radiation received at the detector array is typically dependent upon the attenuation of the x-ray beam by the subject. Each detector element or cell of the detector array produces a separate electrical signal indicative of the attenuated beam received by each detector element. The electrical signals are transmitted to a data processing system for analysis which ultimately produces an image.

Generally, the x-ray source and the detector array are rotated about the gantry within an imaging plane and around the subject. X-ray sources typically utilize x-ray tubes, which emit the x-ray beam at a focal point. X-ray detectors also usually include a scatter-grid for rejecting scattered x-rays at the detector, a scintillator for converting x-rays to light energy adjacent the scatter-grid, and photodiodes for receiving the light energy from the adjacent scintillator and producing electrical signals therefrom. Typically, each scintillator of a scintillator array converts x-rays to light energy. Each scintillator discharges light energy to a photodiode adjacent thereto. Each photodiode detects the light energy and generates a corresponding electrical signal. The outputs of the photodiodes are then transmitted to the data processing system for image reconstruction.

In conventional multi-row CT detectors, a two dimensional array of detector cells extend in both the x and z directions. The active areas of conventional detector cells are generally perpendicular to a plane of x-ray source rotation and, in the context of energy integrating scintillators, convert x-rays to light. The light emitted by each scintillator is sensed by a respective photodiode and converted to an electrical signal. The amplitude of the electrical signal is generally representative of the energy (i.e. the number of x-rays times the energy level of the x-rays) detected by the photodiode. The outputs of the photodiodes are then processed by a data acquisition system for image reconstruction.

Size, shape, and other composition-related concerns place an upper limit on the spatial frequency or sampling resolution which can be achieved by detector arrays. A number of approaches have been developed to overcome the upper sampling limitations of conventional 2D detector arrays. In one proposed solution, miniaturization efforts have led to a reduction in the size of the individual detector cells or pixels. Segmenting the detector active area into smaller cells may increase the Nyquist frequency but can also result in the added expense of more data channels and system bandwidth. Moreover, system detective quantum efficiency (DQE) can be degraded due to reduced quantum efficiency and increased electronic noise which could result in a degradation of image quality.

In another proposed technique, x-ray focal spot deflection or “wobble” has been employed. Deflecting the x-ray focal spot in the x and/or z direction at 2× or 4× the normal sampling rate has been found to provide additional sets of views. For example, in z-wobble techniques, different sets of views are acquired from slightly different perspectives along the z-axis, resulting in unique samples that provide overlapping views of a region-of-interest without subpixellation. This approach typically utilizes a data acquisition system channel capable of very high sampling rates and x-ray source hardware dedicated to rapid beam deflection. However, while the use of x-ray focal spot deflection provides additional unique views, such deflection can present higher power and temperature issues, and uses hardware which may not be found or easily retrofitted into all existing scanners. Moreover, present detectors may not be particularly optimized for receiving deflected x-ray beams and deflected x-ray beams may present image artifacts not normally associated with non-deflection imaging. Additionally, focal spot deflection systems may not preserve x-ray beam interlacing throughout a field of view.

Another proposed approach to increasing sampling density of a CT detector involves the staggering of pixels. Specifically, it is has been proposed that sampling density may be improved by offsetting, in the z direction, every other channel or column of detector cells in the x direction. In one proposed approached, the offset is equal to one-half of a detector cell height. However, this method does not increase the number of samples acquired, and thus does not necessarily reduce aliasing artifacts. And, like the deflection techniques described above, adjusting pixel arrangement requires replacement of existing detectors with new or additional hardware.

Therefore, it would be desirable for an apparatus and method to provide for increased sampling resolution without requiring x-ray deflection or non-conventional detector arrangements. It would be further desirable if such an apparatus and method reduced image artifacts and improved x-ray beam interlacing.

### BRIEF DESCRIPTION OF THE INVENTION

The present invention is directed to a system and method for improving a sampling resolution of a CT imaging procedure through controlling motion of the scan subject. Therefore, embodiments of the present invention overcome the aforementioned drawbacks of focal spot deflection and non-conventional detector arrangements.

In accordance with one aspect of the invention, a CT system includes a rotatable gantry, a subject carrier, a high frequency electromagnetic energy projection source, a scintillator array, a photodiode array, a data acquisition system (DAS), and an image reconstructor. The rotatable gantry has an opening to receive a scan subject translated therethrough by the subject carrier. The high frequency electromagnetic energy projection source is configured to project a high frequency electromagnetic energy beam toward the subject. After passing through the subject, the energy beam is detected by a plurality of scintillator cells of the scintillator array. The photodiode array includes a plurality of photodiodes and is optically coupled to the scintillator array such that the photodiodes detect light output from a corresponding scintillator cell. The photodiode outputs of the photodiode array are received by the DAS. The image reconstructor is connected to the DAS and is configured to reconstruct an image of the subject from the photodiode outputs received by the DAS. The system also includes a computer programmed to increase an apparent axial sampling resolution of the scintillator array by controlling motion of the subject carrier.

According to another aspect of the invention, a method for CT data acquisition is provided. The method includes acquiring CT data during a first rotation of a gantry, then moving a scan subject by a distance corresponding to a factor of a detector cell height. CT data is also acquired during a second gantry rotation and is combined with the data from the first gantry rotation to reconstruct an image with reduced artifacts.

In accordance with a further aspect of the invention, a computer is programmed to sample x-ray detector data during a first rotation of a gantry and sample x-ray detector data during a second rotation of the gantry. The computer also moves a scan subject a given distance to cause paths through a field of view of x-rays detected during the first rotation and paths through the field of view of x-rays detected during the second rotation to be interlaced substantially throughout the field of view.

Various other features and advantages of the present invention will be made apparent from the following detailed description and the drawings.

### BRIEF DESCRIPTION OF THE DRAWINGS

The drawings illustrate one embodiment presently contemplated for carrying out the invention.

In the drawings:

FIG. 1 is a perspective view of a CT imaging system incorporating the present invention.

FIG. 2 is a schematic block diagram of the system illustrated in FIG. 1.

FIG. 3 is a pictorial diagram showing a sampling resolution for a known imaging technique.

FIG. 4 is a pictorial diagram showing a sampling resolution for an imaging technique in accordance with an embodiment of the present invention.

FIG. 5 is a diagram of an x-ray interlacing for a known imaging technique.

FIG. 6 is a diagram of an x-ray interlacing for an imaging technique in accordance with an embodiment of the present invention.

FIG. 7 is a diagram of a helical scanning technique in accordance with an embodiment of the present invention.

FIG. 8 is a diagram of a helical scanning technique in accordance with another embodiment of the present invention.

FIG. 9 is a perspective view of a CT system for use with a non-invasive package inspection system.

### DETAILED DESCRIPTION OF THE PREFERRED EMBODIMENT

The operating environment of the present invention is described with respect to a sixty-four slice computed tomography (CT) system. However, it will be appreciated by those skilled in the art that the present invention is also applicable for use with other multi-slice configurations, such as four, sixteen, thirty-two, and other slice configurations. Moreover, the present invention will be described with respect to the detection and conversion of x-rays. However, one skilled in the art will further appreciate that the present invention is equally applicable for the detection and conversion of other high frequency electromagnetic energy. The present invention will be described with respect to a “third generation” CT scanner, but is equally applicable with other CT systems.

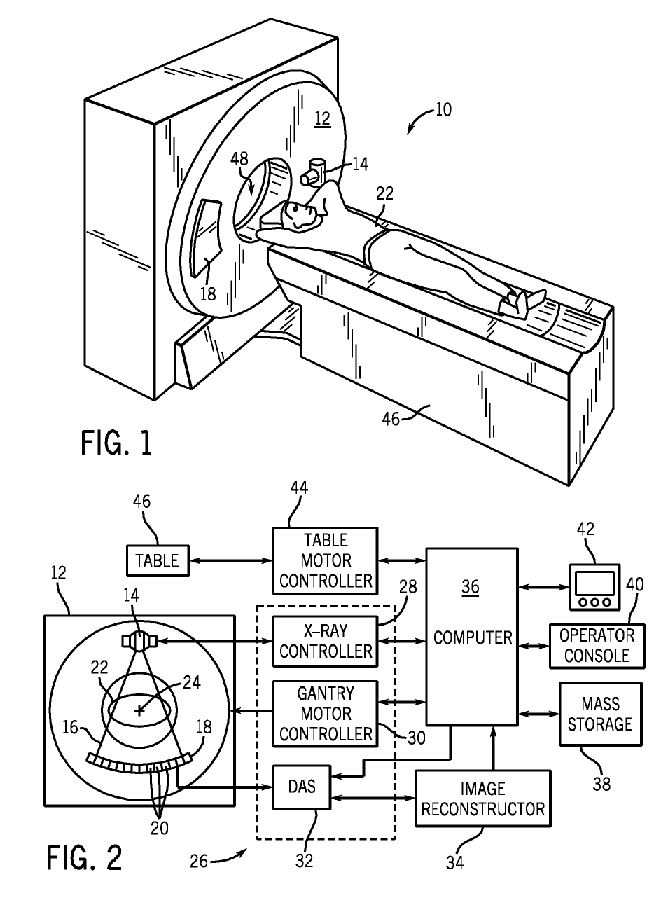

Referring to FIGS. 1 and 2, a computed tomography (CT) imaging system 10 incorporating the present invention is shown. CT system 10 includes a gantry 12 representative of a “third generation” CT scanner. Gantry 12 has an x-ray source 14 that projects a beam of x-rays 16 toward a detector array 18 on the opposite side of the gantry 12. Detector array 18 is formed by a plurality of detectors 20 which together sense the projected x-rays that pass through a medical patient 22. Each detector 20 produces an electrical signal that represents the intensity of an impinging x-ray beam and hence the attenuated beam as it passes through the patient 22. During a scan to acquire x-ray projection data, gantry 12 and the components mounted thereon rotate about a center of rotation 24.

Rotation of gantry 12 and the operation of x-ray source 14 are governed by a control mechanism 26 of CT system 10. Control mechanism 26 includes an x-ray controller 28 that provides power and timing signals to an x-ray source 14 and a gantry motor controller 30 that controls the rotational speed and position of gantry 12. A data acquisition system (DAS) 32 in control mechanism 26 samples analog data from detectors 20 and converts the data to digital signals for subsequent processing. An image reconstructor 34 receives sampled and digitized x-ray data from DAS 32 and performs high speed reconstruction. The reconstructed image is applied as an input to a computer 36 which stores the image in a mass storage device 38.

Computer 36 also receives commands and scanning parameters from an operator via console 40 that has a keyboard. An associated cathode ray tube display 42 allows the operator to observe the reconstructed image and other data from computer 36. The operator supplied commands and parameters are used by computer 36 to provide control signals and information to DAS 32, x-ray controller 28 and gantry motor controller 30. In addition, computer 36 operates a table motor controller 44 which controls a motorized table 46 to position patient 22 and gantry 12. Particularly, table 46 moves portions of patient 22 through a gantry opening 48.

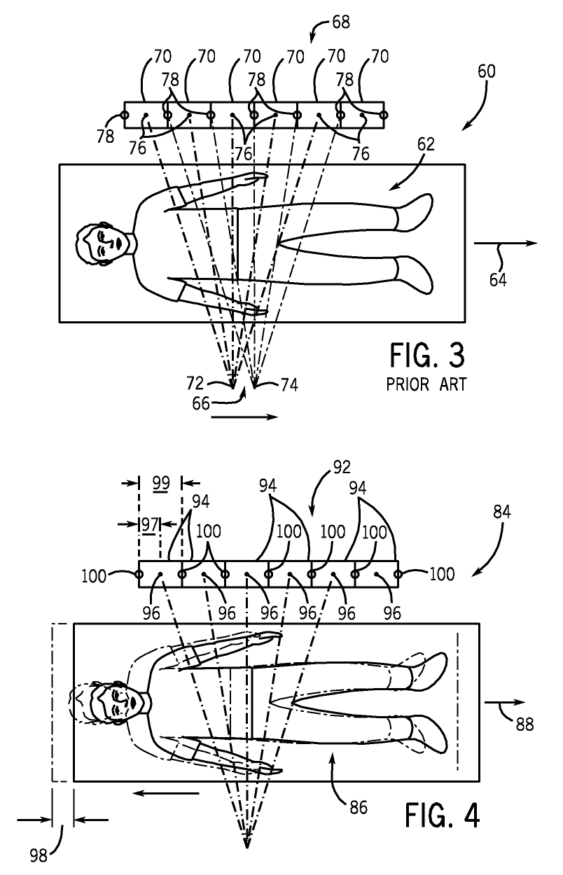

FIG. 3 is a diagram illustrating a technique for improving sampling resolution along the axial or “z” axis through focal spot deflection, commonly known as “z-wobble.” The diagram shows a representation of a simplified CT system 60, including a scan subject 62, an x-ray source 66 and an x-ray detector 68. X-ray detector 68 is shown having a number of detector cells 70 aligned in a single column; however it is to be understood that focal spot deflection is commonly employed with multi row, multi column detectors and that such detectors are typically much smaller in relation to the scan subject 62. As shown, the effective vertex or point of origination of the x-ray fan beam is deflected along the z-axis 64 of the scan subject 62, between a first vertex 72 and a second vertex 74. The distance that the fan beam vertex is deflected is chosen so that the incidence of x-rays upon an imaginary detector at the center of the field of view will be offset between the projections of the first and second vertexes 72, 74 by about half a detector cell height 70 scaled to the center of the field of view. That is, x-rays passing through a given point in scan subject 62 from the first vertex 72 and the second vertex 74 project upon detector 68 at different trajectories. The resulting acquisition from detector 68 is a first set of x-ray data values 76 and a second set of x-ray data values 78 which represent an axially distinct view from the first set. Thus, the effective sampling density in the axial direction, when data from the first and second projections 76, 78 is combined, is increased by a factor of two.

FIG. 4 is a diagram illustrating a technique for improving sampling density in the axial, or “z,” direction according to an embodiment of the present invention. A simplified schematic representation of a CT system 84 includes a scan subject 88 positioned between an x-ray source 90 and an x-ray detector 92. As shown, x-ray detector 92 includes a number of x-ray cells 94, each corresponding to a pixel of an image. However, the features and advantages of the present invention shall be extended to, and are equivalently applicable with, detectors having more cells per row, more rows, etc. In one preferred embodiment, a detector row may have sixty-four detector cells in a row. Additionally, detector cells 94 of detector 92 may be significantly smaller than shown, such as 1 mm in height. In contrast to the technique of FIG. 3, the x-ray source 90 need not be deflected in order to achieve an improved effective number of samples in the axial direction 88; although embodiments of the present invention may be used in combination with x-ray deflection techniques in either or both of the x and z patient axes.

Alternatively, one aspect of the present invention includes shifting a scan subject 86 a distance 98 along the “z” or axial direction 88. X-ray incidence data designated 96 is acquired before the scan subject is shifted the distance 98. X-ray incidence data designated 100 is then acquired after the scan subject has been shifted the distance 98. For representational purposes, data sets 96 and 100 are shown as offset on detector 92. Offset data sets 96, 100 may be acquired for each scan position about a field of view (FOV) as the x-ray source 90 and detector 92 are rotated about the scan subject. In one embodiment, a first axial scan or gantry rotation may be performed to acquire data 96. The scan subject may then be shifted by the distance 98 and a second axial scan or gantry rotation may then be performed to acquire data 100 that is offset from the data of the first axial scan. In effect, by shifting patient 86 the shift distance 98 between scans, an anatomy or point of interest within subject 86 will project upon detector 92 at different points for each acquisition point about the FOV. The result is an increased apparent axial sampling resolution for the desired anatomy in the FOV that is similar to the result achieved by a z-wobble technique.

The distance of the subject shift 98 is preferably determined so that x-rays projected through the same point in a scan subject before and after the shift will be “offset” 97 along the x-ray detector 92 by about one half a height 99 of a detector cell 94. For purposes of illustration, the shift distance 98 is not shown to scale, relative to scan subject 86 and actual detector cell 94 sizes. Moreover, since the actual scan subject shift 98 occurs roughly at the gantry isocenter 88, the subject shift distance 98 should be less than the desired x-ray offset distance 97. That is, because x-ray beams generally propagate in a fan from the source 90, distance measurements at detector 92 are a proportion or factor of equivalent distances at the gantry isocenter 88. For example, if a detector cell 94 is 1 mm in height 99, this may correspond to a height of 0.6 mm after scaling to the isocenter. In this case, the subject would be moved a distance 98 of approximately 0.3 mm or half of the scaled detector size. For many CT systems a distance measurement at the gantry is about 1.7 times the equivalent distance at isocenter. It should be understood that the detector cell measurements and shift distances described herein are by way of example only and that many other detector cell sizes, desired x-ray data offsets, and subject shift distances may be equivalently used. For example, embodiments of the present invention find applicability with detectors having larger cells, and isocenter distance proportions may be larger or smaller according to gantry sizes and x-ray beam fan widths and angles. Likewise, a desired x-ray data offset may correspond to more than or less than half a detector cell height, and/or more than two acquisitions may be overlapped. For example, a desired data offset may be one third of a detector cell height and three acquisitions may be overlapped as such.

Improved sampling resolution may also result in improved image quality by reducing aliasing artifacts, including “bearclaw” artifacts. In addition, the resolution of the reconstructed image in the axial (or “z”) direction may also be improved. That is, by overlapping x-ray data samplings or scans, an increased apparent number of samples in the axial direction results. With more data samples, the effects of aliasing are reduced. Such reduction in the possibility for aliasing artifacts in reconstructed images includes a near elimination of so-called “bear claw” or “pin wheel” aliasing artifacts. Additionally, where an improvement in resolution is not necessarily desired, the x-ray intensity or dosage strength used in the overlapping samplings described above may be reduced. Thus, in embodiments in which data is acquired from two overlapping scans, each scan may be performed at one half the normal x-ray intensity to maintain normal dosage and resolution, but resulting images may be absent bearclaw-type artifacts.

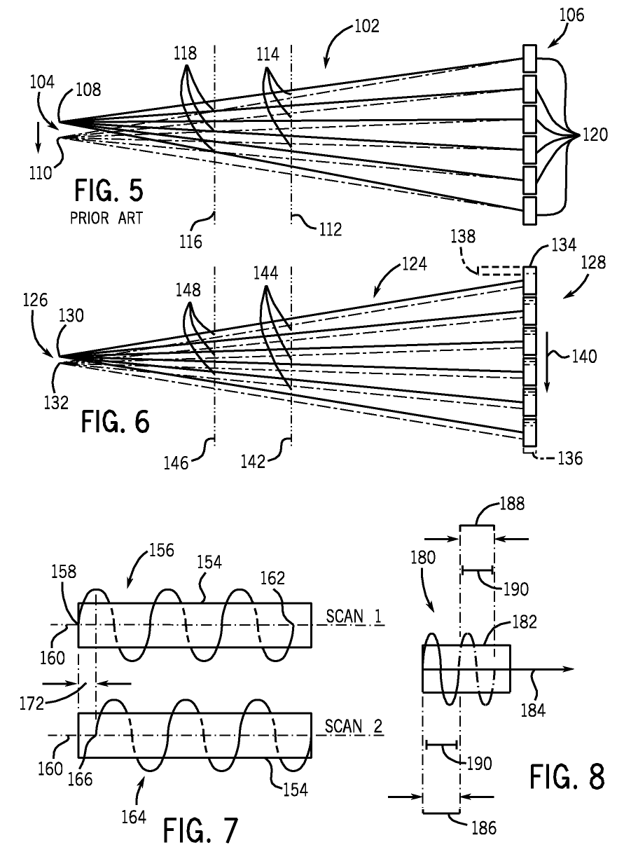

Referring now to FIGS. 5 and 6, an advantage in x-ray interlacing of certain embodiments of the present invention is illustrated. FIG. 5 is a diagram of projected x-rays 102 during a z-wobble imaging procedure. An x-ray source 104 projects x-rays 102 towards one column of a detector array 106. X-rays originate from a first vertex 108 and, after deflection, origination from a second vertex 110. At a gantry isocenter, depicted by line 112, the x-rays of the first projection 108 are well-interlaced 114 with the x-rays of the second projection 110. However, at positions 116 located further from the gantry isocenter 112, the x-rays are not interlaced 118. Therefore, the data acquired at each detector cell 120 for objects at isocenter 112 provides two distinct views, whereas data acquired for objects in the FOV far from isocenter 116 will not provide as much information.

In contrast, FIG. 6 shows a pattern of x-rays 124 which preserves interlacing throughout a FOV 146, 142. X-rays are shown as projected from a source 126 towards one column of a detector array 128. From the perspective of a scan subject being shifted (as discussed above) the x-ray source 126 will appear to project from a first position 130 and a second position 132. However, since it is the scan subject being moved and not the x-ray vertex, x-rays from the first projection 130 will impinge upon the detector 128 at a first position 134 relative to the scan subject and x-rays from the second projection will appear to impinge upon the detector 128 at a second position 136 (shown in phantom) relative to the scan subject. The relative locations of the first x-ray projection 130 and first detector position 134 with respect to the locations of the second x-ray projection 132 and second detector position 136 will depend upon a detector data offset distance 138 which is proportional to the scan subject shift along the z-axis 140. Therefore, x-rays from the first projection 130 and from the second projection 132 have more parallel trajectories than the projections of a z-wobble technique shown in FIG. 5. Accordingly, in the technique of FIG. 6, projected x-rays are well-interlaced 144, 148 at both the gantry isocenter 142 and at positions 146 distant from the isocenter 142, respectively. It is understood, however, that if optimal interlacing is desired at some location other than an isocenter, the amount of subject shift could be varied during or between gantry rotations to adjust the trajectories of the x-rays of the first and second scans.

FIG. 7 depicts an alternative embodiment in which the features and advantages of the present invention are incorporated with a helical scanning technique. Helical scan pattern 156 represents the pattern of x-ray projection view angles relative to a moving scan subject 154. As in most helical scans, the scan subject 154 is translated along the z-axis 160 during data acquisition. Thus, helical scan pattern 156 is shown as beginning at a first point 158 and ending at a second point 162 along the z-axis 160, since the pattern 156 is relative to a moving scan subject 154. According to an embodiment of the invention, a second helical scan 164 may be acquired subsequent to the first helical scan 156 to achieve advantages described above. Thus, second helical scan 164 is shown as beginning at a point 166 with respect to the scan subject 154. Beginning point 166 of second scan 164 is shifted by a distance 172 such that the corresponding source locations throughout first helical scan 156 and second helical scan 164 are offset in the axial or “z” direction by approximately one-half a detector cell height. In practice, a scan subject 154 may be translated along z-axis 160 to acquire data across the desired FOV in a first scan 156, then the scan subject 154 may translated in reverse back to a position 166 which is nearly the same as the original scan start position 158, but offset by an equivalent at isocenter of a desired data offset distance 172. A second helical scan 164 may then be acquired with overlapping data to achieve improved resolution, reduced aliasing artifacts, and/or improved x-ray interlacing, as discussed above.

Similarly, FIG. 8 is a diagram showing another helically-implemented embodiment of the present invention. Rather than acquiring two offset scans as in FIG. 7, the embodiment of FIG. 8 acquires one helical scan at a slower subject translation speed. Thus, during the helical scan sequence 180, the scan subject 182 is translated along the z-axis 184. For each complete gantry rotation 186, 188, the helical scan pattern 180 moves axially by a total of one half a detector cell height 190. In other words, the scan subject 186 is being translated at a constant rate of an isocenter equivalent of one-half a detector cell height 190 per rotation. For detector cells having a 1 mm height, the scan subject would be moved at a rate of about 0.3 mm per rotation. The result is an increased number of samples in the axial direction from interleaved, overlapped scans/rotations. Thus, it is recognized that the features and aspects of the present invention may be achieved in embodiments which acquire data samples via axial scans, multiple helical scans, or single helical scans. The “scans” may each be a single gantry rotation (as is the case for full axial scans), more than one gantry rotation (a helical scan might use 10 rotations or 4.3 rotations), or less than one rotation (“half-scan” axial acquisitions use about ⅔ of a rotation), and may be performed individually or in combination.

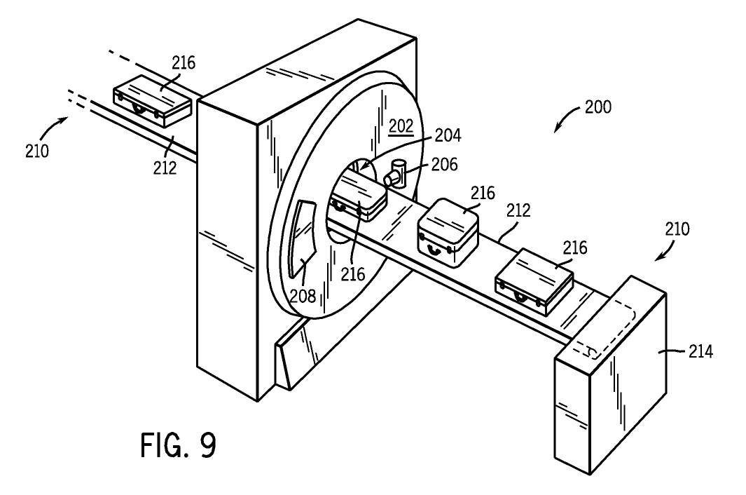

Referring now to FIG. 9, package/baggage inspection system 200 incorporating the present invention is shown. System 200 includes a rotatable gantry 202 having an opening 204 therein through which packages or pieces of baggage may pass. The rotatable gantry 202 houses a high frequency electromagnetic energy source 206 as well as a detector assembly 208 having scintillator arrays comprised of scintillator cells similar to that shown in FIG. 6 or 7. A conveyor system 210 is also provided and includes a conveyor belt 212 supported by structure 214 to automatically and continuously pass packages or baggage pieces 216 through opening 204 to be scanned. Objects 216 are fed through opening 204 by conveyor belt 212, imaging data is then acquired, and the conveyor belt 212 removes the packages 216 from opening 204 in a controlled and continuous manner. Therefore, in incorporating embodiments of the present invention, conveyor belt 212 of package/baggage inspection system 200 (or other package translation mechanisms) may be controlled to provide for increased axial sampling resolution, decreased aliasing artifacts, and improved x-ray interlacing, as discussed above. As a result, postal inspectors, baggage handlers, and other security personnel may non-invasively inspect the contents of packages 216 for explosives, knives, guns, contraband, etc.

Accordingly, a technical contribution of the disclosed method and apparatus is that they provide for a computer implemented CT acquisition technique in which an apparent axial sampling resolution is improved through controlling scan subject motion.

Therefore, in accordance with one embodiment of the present invention, a CT system is provided which includes a rotatable gantry having an opening to receive a subject to be scanned and a subject carrier configured to translate the subject through the opening. A high frequency electromagnetic energy projection source is configured to project a high frequency electromagnetic energy beam toward the subject, to be detected by a scintillator array having a plurality of scintillator cells after the beam has passed through the subject. A photodiode array is optically coupled to the scintillator array and has a plurality of photodiodes configured to detect light output from corresponding scintillator cells of the scintillator array. A data acquisition system (DAS) connected to the photodiode array is configured to receive the photodiode outputs, and an image reconstructor connected to the DAS is configured to reconstruct an image of the subject from the photodiode outputs. The system also includes a computer programmed to increase an apparent axial sampling resolution of the scintillator array by controlling motion of the subject carrier.

According to another embodiment of the invention, a method for CT data acquisition includes acquiring CT data during a first gantry rotation, moving a scan subject by a distance corresponding to a factor of a detector cell height, and acquiring CT data during a second gantry rotation. The data from the first gantry rotation and the data from the second gantry rotation are combined to reconstruct an image with reduced artifacts.

In accordance with a further embodiment of the present invention, a computer is programmed to sample x-ray detector data during a first rotation of a gantry and sample x-ray detector data during a second rotation of the gantry. The computer also moves a scan subject a given distance to cause paths through a field of view of x-rays detected during the first rotation and paths through the field of view of x-rays detected during the second rotation to be interlaced substantially throughout the field of view.

The present invention has been described in terms of the preferred embodiment, and it is recognized that equivalents, alternatives, and modifications, aside from those expressly stated, are possible and within the scope of the appending claims.

What is claimed is:

1. A CT system comprising:
a rotatable gantry having an opening to receive a subject to be scanned;
a subject carrier configured to translate the subject through the opening of the gantry;
a high frequency electromagnetic energy projection source configured to project a high frequency electromagnetic energy beam toward the subject;
a scintillator array having a plurality of scintillator cells wherein each cell is configured to detect high frequency electromagnetic energy passing through the subject;
a photodiode array optically coupled to the scintillator array and comprising a plurality of photodiodes configured to detect light output from a corresponding scintillator cell;
a data acquisition system (DAS) connected to the photodiode array and configured to receive the photodiode outputs;
an image reconstructor connected to the DAS and configured to reconstruct an image of the subject from the photodiode outputs received by the DAS; and
a computer programmed to increase an apparent axial sampling resolution of the scintillator array by controlling motion of the subject carrier.

2. The CT system of claim 1 wherein the computer is further programmed to overlap samplings in an axial direction to reduce artifacts.

3. The CT system of claim 1 wherein the computer is programmed to improve the apparent axial resolution of the scintillator array without applying an x-ray beam deflection.

4. The CT system of claim 1 wherein the computer is further programmed to cause the subject carrier to move an equivalent distance of one-half a sample height of the scintillator array, transposed at isocenter, between corresponding view angles of a first scan and a second scan.

5. The CT system of claim 4 wherein the first scan and the second scan are one of axial scans or helical scans.

6. The CT system of claim 4 wherein the first scan and the second scan comprise one continuous, two rotation scan and the computer is further programmed to cause the subject carrier to move at a rate of the equivalent distance of one-half a sample height per rotation.

7. The CT system of claim 4 wherein the image reconstructor is configured to reconstruct one image having improved resolution from the first scan and the second scan.

8. The CT system of claim 4 wherein the first scan and the second scan are performed at a reduced x-ray dosage.

9. The CT system of claim 1 wherein the computer is further programmed to cause the subject carrier to move a distance to preserve x-ray beam interlacing at non-central locations of the gantry.

10. A method for CT data acquisition comprising:
acquiring CT data during a first scan;
moving a scan subject by a distance corresponding to a factor of a detector cell height;
acquiring CT data during a second scan; and
combining the data from the first scan and the data from the second scan to reconstruct an image with reduced artifacts.

11. The method of claim 10 further comprising determining the offset as a distance equivalent to one half a detector cell height at a gantry isocenter.

12. The method of claim 10 further comprising positioning the first scan and the second scan to overlap a portion of the scan subject to reduce a potential for aliasing in image reconstruction.

13. The method of claim 12 wherein acquiring CT data during the first scan and the second scan includes acquiring CT data during a first helical scan and a second helical scan.

14. The method of claim 12 wherein acquiring CT data during the first scan and the second scan includes acquiring CT data during a first axial scan and a second axial scan.

15. The method of claim 10 further comprising combining the data from the first scan and the data from the second scan to increase an apparent sampling resolution in an axial direction.

16. The method of claim 10 further comprising preserving x-ray interlacing between the first scan and the second scan throughout a field of view.

17. A computer programmed to:
sample x-ray detector data during a first scan;
sample x-ray detector data during a second scan; and
move a scan subject a given distance to cause paths through a field of view of x-rays detected during the first scan and paths through the field of view of x-rays detected during the second scan to be interlaced substantially throughout the field of view.

18. The computer of claim 17 further programmed to determine the distance to move the scan subject to increase a number of x-ray detector data samples in an axial direction for an image reconstructed from the x-ray detector data of the first and second rotations.

19. The computer of claim 17 further programmed to determine the given distance to move the scan subject to reduce aliasing artifacts in an image reconstructed from the x-ray detector data of the first and second rotations.

20. The computer of claim 17 wherein the first scan includes one of a first axial scan and a first helical scan and the second scan includes one of a second axial scan and a second helical scan offset by the given distance the scan subject is moved.

21. The computer of claim 17 wherein the first and second scans comprise a continuous helical scan and wherein the scan subject is moved at a rate of one-half the given distance per rotation.

22. The computer of claim 17 wherein the given distance corresponds to one-half a detector cell height at an isocenter of the gantry.

23. The computer of claim 17 further programmed to cause x-rays projected during the first and second rotations to have an aggregate dosage to achieve a desired resolution in an image reconstructed from the data sampled during the first and second rotations.
